(19) United States
(12) Patent Application Publication
(10) Pub. No.: US 2005/0113641 A1
(43) Pub. Date: May 26, 2005

(54) Endoscopic imaging and intervention system

(76) Inventor: John L. Bala, Pomfret Center, CT (US)

Correspondence Address: ALIX YALE & RISTAS LLP, 750 MAIN STREET, SUITE 1400, HARTFORD, CT, 06103, US

(21) Appl. No.: 10/718,771

(22) Filed: Nov. 22, 2003

(51) Int. Cl. A61B 1/04; A61B 1/06

(52) U.S. Cl. 600/108; 600/104; 600/132

(57) ABSTRACT

An endoscope system for providing imaging and intervention therapy comprises a control module, a light source in electrical communication with the control module, an intervention energy source in electrical communication with the control module, and a micro-endoscopic device. The micro-endoscopic device includes an optical probe, in optical communication with the light source and the intervention energy source, and a sensor array, in optical communication with the optical probe and in electrical communication with the control module. The light source is activated by the control module to emit a series of light pulses which are directed to a target area by the optical probe. Light reflected from the target area is collected by the optical probe, which conducts the reflected light to the sensor array. The sensor array transmits an image of the target area to the control module. The control module is operable to activate the intervention energy source to emit intervention energy which is conducted to the target area by the optical probe.

### BACKGROUND OF THE INVENTION

This invention relates generally to endoscopes which are used in the field of medicine. More particularly, the present invention relates to endoscopes including apparatus for providing medical intervention.

Originally developed in 1966 by Dr. H. H. Hopkins, the rigid endoscope has changed little in basic form and design, generally having a diameter of 10 mm and a length of 350-400 mm. The Hopkins design included non-imaging fiber optics for illumination conduits and optics having a telescopic design that is focused at infinity, thus enabling the endoscope to remain in focus throughout its operating range. An objective lens captures the image from inside the body and relays it through a series of rods and lenses to convey the image from the objective elements to an eyepiece at the end of the endoscope. The system operates in a manner such that as light exits one rod element, a collimating lens intercepts and re-directs the optical rays to a second rod, and, then, to a third rod lens and so forth until the image has been conveyed to an eyepiece at the end of the device. Additional lenses located in the eyepiece capture the image from the final relay system and focus it on the human eye.

Such “rigid” scopes are used to perform procedures within the trunk of the body. Rigid surgical endoscopes enter the body through relatively large incisions made by a trocar or a cannula. Pelvic examinations, tubal ligation, and gall bladder removals were among the first surgical procedures using endoscopes to be approved by the FDA, and remain among the most often performed surgeries. Due to the potential for bleeding, pain and trauma, procedures utilizing rigid endoscopes are preformed in a hospital or clinic and generally necessitate either local or general anesthesia. Accordingly, the entire process is both traumatic and expensive for the patient.

A smaller version of the Hopkins design, an arthroscope, is 2.7 mm in diameter and 150-200 mm in length. The principles of imaging are the same, only the applications are different. Because smaller diameters minimize trauma to human tissue, orthopedic specialists currently use these smaller diameter scopes to diagnose and treat injuries. More than 2.7 million arthroscopic procedures are performed annually. These include over 1.7 million knee procedures, 500,000 shoulder injuries, 200,000 elbow, ankle and wrist injuries, and 200,000 spinal procedures.

Much of the early development in medical fiber optics for flexible endoscopes was performed at the American Optical Corporation (AOC). AOC pioneered the combining of thousands of individual, spatially aligned fibers that are needed to relay an image along the length of the fiber. AOC's early research created fiber optic illumination systems, coherent fiber imaging and remote articulation technologies that are still the basis of all modern flexible endoscopes. Flexible endoscopes require objective lenses with optical designs that are different for each fiber diameter, field of view, or working distance. These designs are optimized for a given application along with the illumination conduits. The imaging fibers are equivalent to the rod lenses in the rigid scope, but are capable of conveying the image over much longer lengths without significant transmission loss. However, as a general rule performance of the fiber optics increases with diameter and decreases with length while versatility of the fiber optics decreases with diameter and increases with length.

Both rigid and flexible endoscopes require the use of illumination conduits to provide light for illuminating the interior of the patient's body. From a practical and functional perspective, glass fiber is generally the best material for such use. Fiber optic light guides can transmit light frequencies from the ultra violet to the near infrared spectrum. They are relatively non-expensive, durable, optically efficient and functional. Glass can also withstand frequent sterilization by autoclaving and other standard methods.

Current illumination systems for medical applications utilize continuous (CW) light sources which are coupled to the illumination conduit by a light guide and an optical connector located at or near the top of an endoscope. In medical endoscopes, tungsten, halogen, deuterium and CW xenon are the most common light sources that are used. These lamps possess a finite lifetime (approximately 500 to 1000 hours) and can fail during surgical procedures. Therefore, dual high wattage (150-300 watts) lamps and power supplies are used to insure availability of a second source should a lamp fail during a procedure. The light output of these lamps constantly deteriorates and the lamps often require warm-up time prior to operating in a stable manner. Endoscopic CW illumination systems cannot share their optical paths with laser intervention devices, as these paths are continually required for imaging. Furthermore, a laser used in the visible spectrum would convolute the image creating a color imbalance or distortion as well as saturating the camera system.

The connector fibers at the input of the endoscope do not align directly with the cable fibers which reduces coupling efficiency and causes heating of the junction of the cable and connector on the scope. CW light sources also generate considerable heat at the surgical site when the distal end of the device is moved within close proximity to living tissue. This can create a problem of changing coagulation at the surgical site. In addition, endoscopes placed near the patient in preparation for surgery have generated sufficient heat such that disposable paper drapes used to cover patients have caught fire. All these situations place the patient at considerable risk

For many years, endoscopes have been used to provide images from within the human body for viewing by a doctor through an eyepiece. In more recent times, a digital camera with ever-increasing resolution and image quality has replaced direct viewing with the eye. As the sophistication of camera systems has increased, so has the cost of such systems with the camera systems of current endoscopes comprising the major cost element of such devices.

As doctors have demanded higher quality images from within the body, new charged coupling device (CCD) camera technology utilizing three chip cameras with high resolution have become the method of choice. Three chip medical cameras utilize the entire active surface area of a single camera for each color. Thus, three monochrome cameras are used, one with a red filter, one with a green filter, and one with a blue filter. A beam splitter splits the light from the image, providing simultaneous input into each of the cameras. Three chip camera systems cost between $15,000 to $50,000 depending on options, features and capability. The high cost of the three cameras and associated electronics represents a substantial cost penalty for these systems. A technical penalty results from sharing the image plane, thus reducing the amount incident on each camera. Other surface reflection losses further reduce the amount of light incident on the camera which, in small diameter light-starves systems, is a further disadvantage.

The use of lasers for surgical intervention represents both a vast, complex problem and a unique opportunity. To date, thousands of publications have been written about both endoscopic and laser surgery, the standardization of laser frequencies, the diameters of various delivery systems, spot size, contact or non-contact application means, power levels, side effects, etc. Simply stated, the proper selection of the various parameters allows a surgeon to control a heat and/or chemical reaction induced by the laser. To combine image-guided endoscopy with laser intervention, it is necessary to understand how optical systems control laser parameters in order to provide the most effective management of heat and/or chemical reactions. The compatibility of technology used for imaging and for intervention must also be insured. The optical effects of focused or defocused beams creates either convergence or divergence at surgical site. Focused light is used to create vaporization, while a defocused beam may be used for coagulation. Laser power is also a major consideration, as even a focused beam of low-powered laser light can create a significant amount of heat at its focal point.

### SUMMARY OF THE INVENTION

Briefly stated, the invention in a preferred form is an endoscope system for providing imaging and intervention therapy which comprises a control module, a light source in electrical communication with the control module, an intervention energy source in electrical communication with the control module, and a micro-endoscopic device. The micro-endoscopic device includes an optical probe, in optical communication with the light source and the intervention energy source, and a sensor array, in optical communication with the optical probe and in electrical communication with the control module. The light source is activated by the control module to emit a series of light pulses which are directed to a target area by the optical probe. Light reflected from the target area is collected by the optical probe, which conducts the reflected light to the sensor array. The sensor array transmits an image of the target area to the control module. The control module is operable to activate the intervention energy source to emit intervention energy which is conducted to the target area by the optical probe.

The light source comprises a plurality of flashtube assemblies, each of which includes a xenon flashtube and a color separation filter disposed downstream of the output face of the flashtube. Preferably, the light source comprises three flashtube assemblies, with the color separation filter of the first flashtube assembly being a red filter, the color separation filter of the second flashtube assembly being a green filter and the color separation filter of the third flashtube assembly being a blue filter.

A first service cable assembly electrically connects the control module to the light source and the intervention energy source. A second service cable assembly includes an electrical conduit and an optical conduit, electrically and optically connecting the light source and the intervention energy source to the micro-endoscopic device. An infrared filter is disposed within the light path of the optical conduit.

In a first embodiment, the second service cable assembly comprises a conduit segment and an input segment including a trifurcated fiber optic conduit having three input ends, with one of the input ends being in optical communication with each of the flashtube assemblies. An ultra violet filter is movable from an imaging position, within the light path of the optical conduit, to a intervention position, outside of the light path of the optical conduit.

In a second embodiment, the second service cable assembly comprises a conduit segment and an input segment including a quad-furcated fiber optic conduit having four input ends. The first, second and third input ends are in optical communication with the first, second and third flashtube assemblies, respectively. The fourth end is in optical communication with the intervention energy source, which is a therapeutic laser unit. The input segment also includes a mechanical fail-safe shutter disposed between the laser unit and the fourth input end. The shutter is in electrical communication with the control module, whereby the control module maintains the shutter in a closed position except when intervention therapy is administered. The sensor array also has a shutter in electrical communication with the control module, with the shutter being disabled in a closed position when intervention therapy is administered.

In a third embodiment, the input segment of the second service cable assembly comprises a trifurcated fiber optic conduit and a bifurcated fiber optic conduit, the trifurcated fiber optic conduit having first, second and third input ends in optical communication with the first, second and third flashtube assemblies, respectively. The bifurcated fiber optic conduit has a fourth end in optical communication with a fourth flashtube assembly and a fifth end in optical communication with the intervention energy source. The intervention energy source is a therapeutic laser unit and the system further comprises an optical connector disposed between the fifth end and the laser unit. The optical connector is turret-mounted optics providing selection of different laser effects.

It is an object of the invention to provide a new and improved endoscope system for providing imaging and intervention therapy.

It is also an object of the invention to provide a new and improved endoscope system having multiple xenon flashtubes for imaging and intervention therapy.

It is further an object of the invention to provide a new and improved endoscope system having multiple xenon flashtubes for imaging and a laser for intervention therapy.

Other objects and advantages of the invention will become apparent from the drawings and specification.

### BRIEF DESCRIPTION OF THE DRAWINGS

The present invention may be better understood and its numerous objects and advantages will become apparent to those skilled in the art by reference to the accompanying drawings in which:

FIG. 1 is a simplified perspective view of a first embodiment of an endoscopic imaging and intervention system in accordance with the present invention;

FIG. 2 is a simplified perspective view of a second embodiment of an endoscopic imaging and intervention system in accordance with the present invention;

FIG. 3 is a simplified perspective view of a third embodiment of an endoscopic imaging and intervention system in accordance with the present invention;

FIG. 4 is an enlarged perspective view of the micro-endoscopic device of FIG. 3; and

FIG. 5 is a graphical illustration of the energy with respect to time of a pulse of light emitted by a xenon flashtube appropriate for use in conjunction with an endoscopic imaging and intervention system in accordance with the present invention.

### DETAILED DESCRIPTION OF THE DRAWINGS

With reference to the drawings wherein like numerals represent like parts throughout the several figures, a system in accordance with the invention may be used for both endoscopic imaging and endoscopic intervention therapy.

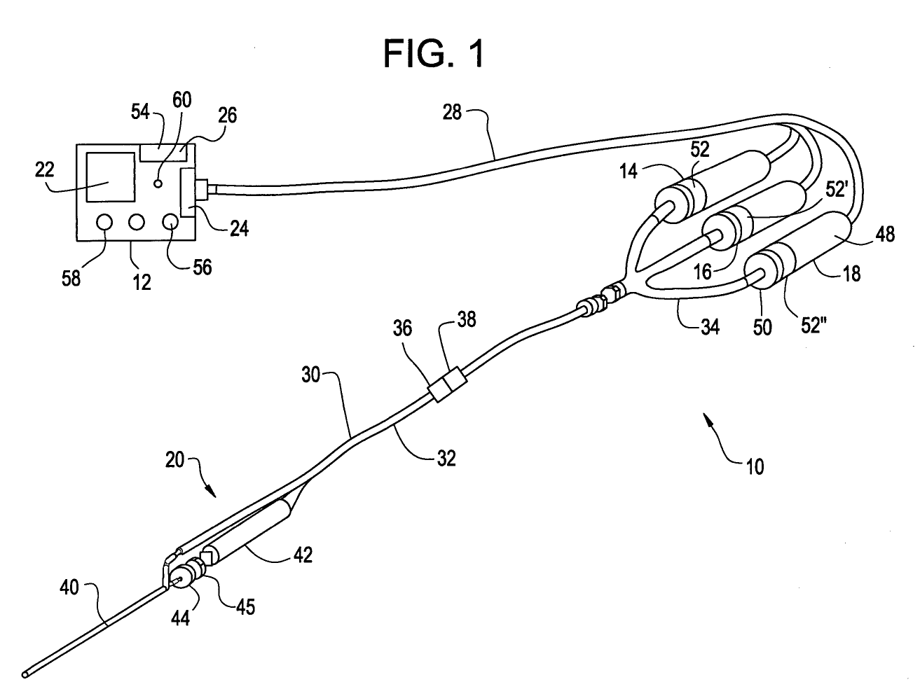

A first embodiment of an endoscopic imaging and intervention system in accordance with the present invention is generally designated by the numeral 10. With reference to FIG. 1, the endoscopic imaging and intervention system 10 includes a control module 12, three flashtube assemblies 14, 16, 18, and a micro-endoscopic device (MED) 20. The control module 12 includes a video monitor 22, system power supplies 24, and a system processing and control electronics unit 26. A first service cable assembly 28 electrically connects the control module 12 to the three flashtube assemblies 14, 16, 18 and a second service cable assembly 30 electrically and optically connects the three flashtube assemblies 14, 16, 18 to the MED 20. The second service cable assembly 30 includes a conduit segment 32 and an input segment 34, having a trifurcated fiber optic conduit which multiplexes or randomizes the light output of each flashtube assembly 14, 16, 18 to produce a single light output to the conduit segment 32. Conventional systems use continuous wave (CW) illumination systems that monopolize the fiber optics which channel light into the body. Multiplexing the illumination light allows the fiber optics to be used for both illumination and image transmission.

The second service cable assembly 30 is similar in many respects to the service cable disclosed in U.S. patent application Ser. No. 09/754,892, hereby incorporated by reference. More specifically, the second service cable assembly 30 includes a fiber optic bundle to transmit light from the light source to the MED 20. An infrared (IR) filter 36 and a removable ultra violet (UV) filter 38 is provided to remove IR and UV energy from the light carried in the fiber optic bundle during imaging to protect body tissue. The UV filter 38 is removed for intervention therapy, to harness the UV energy, as explained in greater detail below. The second service cable assembly 30 also incorporates electrical conductors to allow the control module to communicate with the electronic portions of the MED 20.

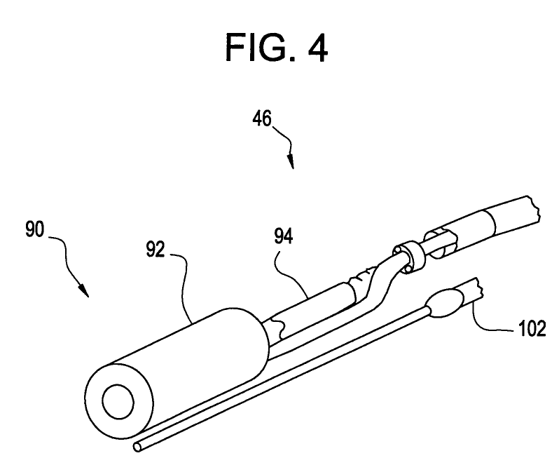

The MED 20 may also be similar to the MED disclosed in U.S. patent application Ser. No. 09/754,892. More specifically, the MED 20 may comprise an optical probe 40, a CCD sensor array 42 (which may also be referred to as a camera), and imaging lenses 44. The optical probe 40 acts as a fiber optic conduit as both an illumination conduit, providing light for illuminating the interior of the patient's body, and an imaging conduit, providing a path for images of the interior structures of the patient's body. Flexible or rigid optical probes having a 0.5-1 mm diameter may be used. A UV filter 45 is positioned between the optical probe 40 and the CCD sensor array 42 to protect the array 42 during intervention therapy. Preferably, the MED 20 also comprises a sensor head which contains a zoom/image focus optics package and a light pulse transfer interface. Alternatively, an MED 46 in accordance with FIG. 4 (described in detail below) may be used.

Each flashtube assembly 14, 16, 18 includes a pulsed xenon flashtube 48 which emits a pulse of light of great intensity and broad spectrum but extremely short duration. For example, the flash tube 48 may emit a light pulse having the equivalent of 100,000 watts of light power, but lasting only 10 microseconds. A continuous source of light having this intensity would generate significant and unwanted quantities of heat. The short duration of the light pulses from the flash 48 tube avoids any,significant heat buildup. Light generated by the flashtube 48 is focused on the light receiving face of one of the three fiber optic illumination channel of the trifurcated fiber optic conduit 34 by light focus optics 50. The light focus optics 50 further enhance the intensity of light incident on the receiving face by gathering, directing and focusing the light. A color separation filter 52 is located between the output face of the flashtube 48 and the input face of the light focus optics 50. The color separation filter 52 is a band-pass filter, allowing only a narrow frequency band of light to pass into the fiber optic 34. Preferably, the color separation filter 52 of the first flashtube assembly 14 is a red filter, the color separation filter 52′ of the second flashtube assembly 16 is a green filter and the color separation filter 52″ of the third flashtube assembly 18 is a blue filter. Specialized filters 52 may be substituted for those designed for use in the visible spectrum.

Software 54 stored in the memory of the system processing and control electronics unit 26 controls the video frame rate, pulse, intensity, and camera shutter and generates the full color images. In addition, the software 54 controls the monitor display 22 for the instrument set-up status. The status information is removed once appropriate conditions have been established. The software 54 may also control printers and a RD/WR compact disc recorder.

For imaging, the system processing and control electronics unit 26 directs the power supply 24 to selectively transmit pulses of electrical power to the flashtubes 48, causing the flashtube assemblies 14, 16, 18 to transmit sequenced, red, green and blue (RGB) light pulses into the patient's body via trifurcated fiber optic conduit 34, the fiber optic conduit 32 of the second service cable assembly 30, and the MED optical probe 40. Each flashtube's power output is voltage-controlled. An increase in the control voltage increases light output and, consequently, the brightness of the video image. A reduction in voltage to the pulse xenon circuitry reduces the brightness. The image of the area of treatment, comprised of pulses of RGB light reflected from the organ(s) within the area of treatment, is collected by the MED optical probe 40 and transmitted therethrough to the CCD sensor array 42. The monochrome CCD sensor array 42 then processes the pulses one at a time, a frame grabber converts the analog image to a digital image and transmits the digital image to a buffer in the system processing and control electronics unit 26, where the digital image is processed to create a high resolution color video image of the area of treatment.

The system 10 generates RGB light pulses in continuous bursts of three to sixty pulses per flashtube 48 per second (sixty pulses per second being the design maximum). In operation, however, the practical pulse rate for providing updated video images is 20-60 pulses per second for each flashtube 48, with a pulse width of 8 microseconds. The camera 42 sequencing captures the image, frame by frame, from each pulse, and stores each image in memory in the system processing and control electronics unit 26. The system processing and control electronics unit 26 then combines the three frames to create a composite video image on the monitor 22. A composite video image is sent to the monitor 22 after each set of light pulses is processed. A control 58 on the control module 12 allows the pulse rate to be varied within a selected range in order to optimize the video image for a given procedure. The RGB light pulses are approximately 8-12 microseconds out of a duty cycle of 33 milliseconds. This allows the remainder cycle to be used in performing intervention or pulsed imaging.

There are many advantages to using the system 10. The system 10 provides a higher contrast image at the same resolution as a three chip camera system at about half the cost. It has almost infinite life and replacement parts are rarely needed. More importantly, the long life means that there is little possibility of lamp failure during diagnostic or surgical procedures. The system 10 generates practically no heat in either the patient or the instrument. Perhaps, most important, it allows sharing of the light entering the body for intervention as well as imaging.

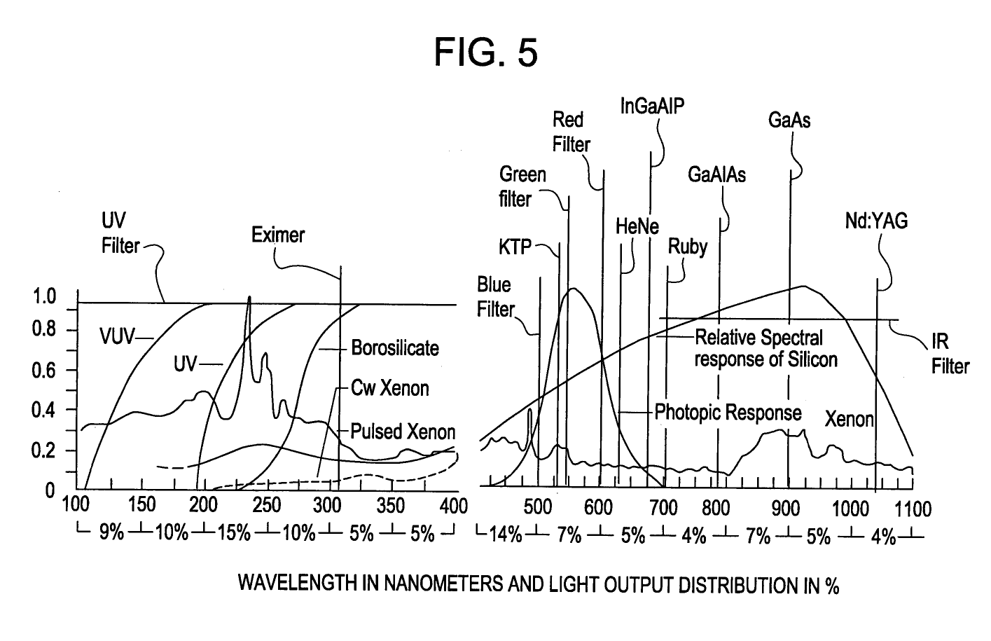

FIG. 5 shows the optical spectrum for pulsed xenon and the wavelengths of various therapeutic lasers. This graph shows the flashtube output being extremely rich in UV and having high energy content in the visible and infrared parts of the spectrum. The graph includes the spectral distribution of the flashtube and its window transmittance. For imaging, optical filters 38, 36 remove both the ultra violet and infrared light. The imaging wavelengths are based on studies performed by DeMarsh for the National Bureau of Standards for Color Television. The wavelengths for the red, green and blue color separation filters 52, 52′, 52″ are 650 nm, 550 nm, and 435 nm, respectively. These wavelengths create a type of abridged spectrophotometer, and are consistent with CIE color reproduction criteria. Each color separation filter 52, 52′, 52″ has an optical bandwidth of 10-20 nm. These design parameters were selected to produce the best reproduction of color images on a video monitor 22. Other color filters can provide the same function.

For intervention therapy, the UV filter 38 is removed and all three flashtubes 48 are triggered simultaneously at high levels of energy to emit UV energy. The flashtube UV energy, which has a major peak at 230 nm, provides non-coherent light from 150-350 nm to create a non-ablative photochemical reaction. The optical probes small diameter allows a physician to pinpoint an area at which to deliver a bolus of UV energy from all three flashtubes 48 firing at the same time. The bolus triggers an interaction that may kill infection, seal a wound, open a passageway or dissolve a polyp, for example.

A key switch 60 on the control module 12 allows the surgeon to activate a program stored in the system processing and control electronics unit memory 26. The program triggers the flashtubes 48 with multiple power pulses at increasing pulse rates and/or increasing voltage to raise the UV energy level at the treatment site to a predetermined value stored in the memory. The system 10 continues to generate pulses as long as the key switch 60 is held closed. Internal images displayed on the video monitor 22 provide in situ images interspersed with information such as the pulse frequency, the pulse energy setting, and the total number of UV pulses transmitted.

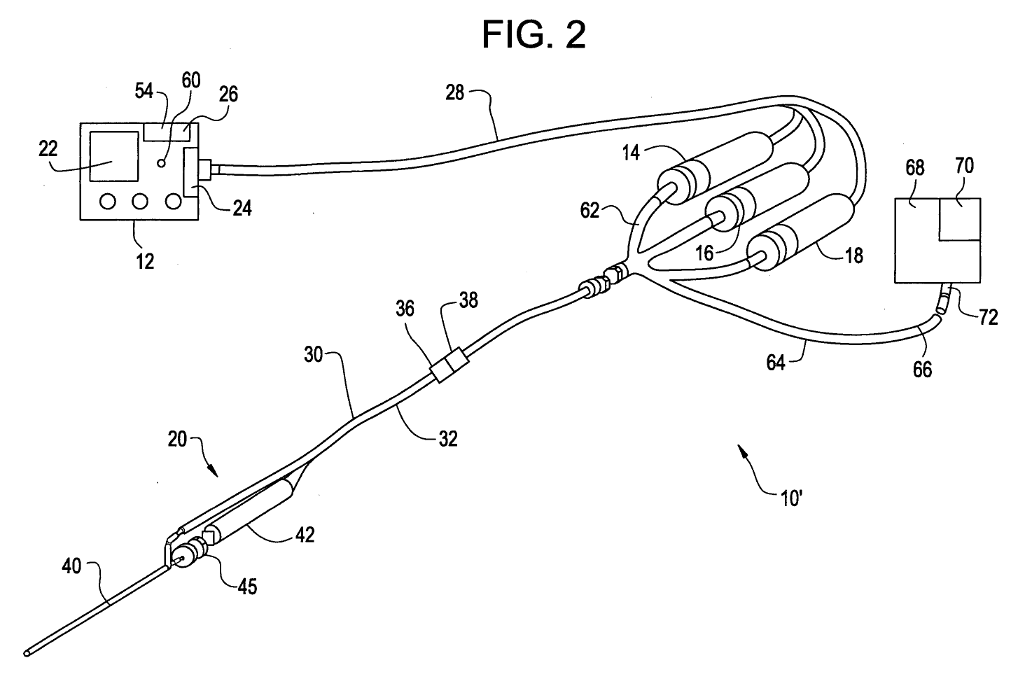

A second embodiment of an endoscopic imaging and intervention system in accordance with the present invention is generally designated by the numeral 10′. With reference to FIG. 2, the trifurcated fiber optic conduit of the first embodiment 10 is “expanded” to a quad-furcated fiber optic conduit 62 having a fourth fiber optic illumination channel 64. An optical connector 66 on the input of the fourth fiber optic illumination channel 64 provides an interface for a therapeutic laser unit 68, allowing the coherent laser light to be directed into the body cavity.

Software 54 stored in the memory of the system processing and control electronics unit 26 directs operation of the flashtube assemblies 14, 16, 18, the laser unit safety controls 70, which prevent inadvertent exposure from the laser 68 and UV from the flashtubes 48 to the patient and any attending medical personnel. A mechanical fail-safe shutter 72 positioned between the laser unit 68 and the fourth fiber optic illumination channel 64 blocks the laser light, preventing it from entering into the fourth fiber optic illumination channel 64, and subsequently the body. The shutter 72 remains closed unless opened by the software 54. The camera shutter itself is disabled electronically to prevent visible laser sources from saturating the camera system 42. The software 54 also provides sequential control of the flashtubes 48 and laser unit 68 for imaging or for laser for intervention therapy.

The laser can be used with micro probes in either non-contact vaporization mode or coagulation mode. The laser can be structured to provide either a divergent beam or a convergent, focused beam. Its power setting is increased for vaporization and reduced for coagulation. For interstitial tissue coagulation, relatively long application times (5-20 minutes) are used at low power settings (4-10 watts) in continuous mode. For vaporization, power settings of 8-12 watts of focused energy are necessary. The working distance is approximately 5-10 mm from the tissue.

Laser systems which may be used are: Ruby, 694 nm; Nd:YAG, 1064 nm; Ho:YAG, 2130 nm; KTP, 532 nm; Alexandrite, 720-800 nm; GaAs, 904 nm; GaAIAs, 780-820-870 nm; InGaAIP, 630-685 nm; Rhodamine, 560 nm; HeNe, 633 nm; Argon, 350-514 nm; Eximer, 193-248-308 nm; and Copper Vapor, 578 nm. The CO² laser at 10,600 nm and the Er:YAG laser at 2940 nm cannot be used with the system 10′ because their wavelengths are beyond the limit of fiber optic transmission for normal fibers. The laser light pulses would be interspersed with the RGB light pulses and will not interfere with imaging. Although several laser wavelengths are close to the imaging wavelengths (650 nm, 550 nm, and 435 nm), the RGB color separation filters will insure that no overlap occurs either at the natural or harmonic frequencies.

An ablation laser can be used in conjunction with UV light from the flashtube assemblies 14, 16, 18 to provide photo coagulation in a post-ablation procedure. This is accomplished by removing UV filter 38, simultaneously pulsing the three flashtubes to produce a bolus of UV energy, and interspersing the bolus of UV energy with laser pulses. In this application, UV could create coagulation and the laser, photothermal ablation. Software 54 stored in the system processing and control electronics unit 26 directs operation of the flashtube assemblies 14, 16, 18 and the laser unit 68 to create this effect. Imaging, UV intervention therapy and laser intervention are, thus, all interwoven together by multiplexing the fiber optic system. This combination of technologies into a single device is possible because of the dynamic pulsed system, it is not possible with conventional, static imaging systems.

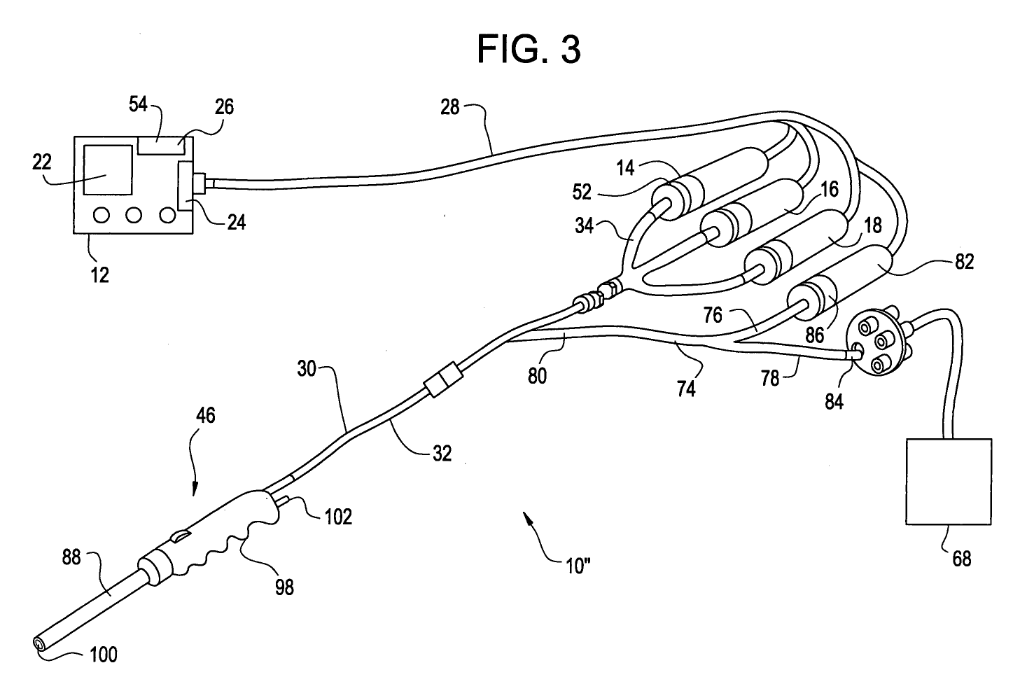

A third embodiment of an endoscopic imaging and intervention system in accordance with the present invention is generally designated by the numeral 10″. With reference to FIG. 3, this embodiment 10″ integrates endoscopic imaging with a therapeutic laser interface designed for image-guided intervention. The laser interface in this embodiment 10″ comprises a bifurcated fiber optic conduit 74, having fourth and fifth fiber optic illumination channels 76, 78 and an output 80 coupled to the second service cable assembly 30. A fourth flashtube assembly 82 is optically coupled to the fourth fiber optic illumination channel 76 and an optical connector 84 on the input of the fifth fiber optic illumination channel 78 provides an interface for a therapeutic laser unit 68. The bifurcated fiber optic conduit 74 multiplexes or randomizes the non-coherent light output of the fourth flashtube assembly 82 and the coherent light output of the laser unit 68 to produce a single light output to the conduit segment 32 of the second service cable assembly 30. The software 54 stored in the system processing and control electronics unit 26 directs the multiplexing through the bifurcated fiber optic conduit 74. The software 54 also causes the marker flashtube assembly 82 to paint the anatomical target onto the video monitor 22, differentiating the marker channel 79 from the imaging channels 34, and clearly marking the area of laser treatment.

Since many lasers are outside of the visible spectrum, the fourth flashtube assembly 82 having a narrowband optical filter 86 in the visible area of the spectrum is used for creating the marker for laser targeting. The optical filter 86 of the fourth flashtube assembly 82 is for a different wavelength than the filters 52, 52′, 52″ of the first, second and third flashtube assemblies 14, 16, 18. The image from this flashtube assembly 82 is projected onto tissue that the laser will irradiate. The target is overlapped by the laser position as viewed on a video monitor 22 along with the endoscopic image. An additional sequence of the CCD camera system 42 reads an image of the targeted area, and integrates the display of the marked site with the image extracted from the body. Software 54 stored in the system processing and control electronics unit 26 process this information to differentiate the video image from the target on the video monitor 22.

Preferably, the optical connector 84 on the input of the fifth fiber optic illumination channel 78 comprises turret-mounted optics, allowing the selection of different laser effects (i.e. ring-mode applicators, convergent beams, divergent beams, and scattering applicators) that will create different patterns of laser photothermal ablation or photocoagutatlon. The selection of optics at the input of the fifth fiber optic illumination channel 78 allows for a selection of collimated, divergent, convergent, ring and scattering applicators for interstitial therapy. The ring applicator is used for treatment of tumors. The scattering applicator can spread lower power over the emitted surface. The three pulse imaging system allows real-time monitoring and visualization of the procedure in situ with intervention performed in a non-contact mode.

With reference to FIG. 4, the preferred MED 46 for the third embodiment 10″ comprises an optical probe 88, a sensor head 90 which contains a zoom optical system 92, the CCD sensor array 94 and a light pulse transfer interface. An MED housing 98 is a rigid structure which may be integrally connected to the optical probe 88. The housing 98 has a compact hand-held configuration that is exteriorly contoured to fit the hand of a user to facilitate dexterous and versatile usage. The optical probe 88 is tubular, having an internal channel 100 for injecting saline or gas for rinsing or cooling the surgical site. A luer lock 102 prevents the fluid or gas from escaping from the MED 46.

The zoom optical system 92 is preferably a three-or-four position fixed zoom. Each of the zoom positions is pre-focused at each magnification. Current instruments can only generate a zoom equivalent when the surgeon inserts the probe deeper into the surgical site to increase magnification, or moves the probe away to increase the field of view. The internal optics of the subject zoom optical system 92 create the zoom, not movement of the endoscope. The pre-focused, fixed zoom positions eliminate the need to move the endoscope in and out in order to achieve changes in magnification or field of view. This is especially important during laser treatment, as it is critical that the focal point of the laser or pattern used for coagulation remain constant.

While preferred embodiments have been shown and described, various modifications and substitutions may be made thereto without departing from the spirit and scope of the invention. Accordingly, it is to be understood that the present invention has been described by way of illustration and not limitation.

1. An endoscope system for providing imaging and intervention therapy comprising:
a control module;
a light source:
an intervention energy source;
a micro-endoscopic device;
a first service cable assembly electrically connecting the control module to the light source and the intervention energy source; and
a second service cable assembly electrically and optically connecting the light source and the intervention energy source to the micro-endoscopic device;
the micro-endoscopic device including
a sensor array in electrical communication with the control module,
an optical probe in optical communication with the second service cable assembly and the sensor array, and
an object lens in optical communication with the optical probe;
wherein the light source is activated by the control module to emit a series of light pulses, the second service cable assembly conducts the light pulses to the optical probe, the optical probe directs the light pulses to a target area, the object lens collects light reflected from the target area, the optical probe conducts the reflected light to the sensor array, and the sensor array transmits an image of the target area to the control module, and wherein the control module is operable to activate the intervention energy source to emit intervention energy which is conducted to the target area by the second service cable assembly and the optical probe.

2. The endoscope system of claim 1 wherein the control module comprises:
a video monitor;
system power supplies; and
a system processing and control electronics unit.

3. The endoscope system of claim 2 wherein the system processing and control electronics unit comprises:
a memory and
software stored in the memory, the software controlling the video monitor, the system power supplies, the intervention energy source and the micro-endoscopic device.

4. The endoscope system of claim 1 wherein the light source comprises a plurality of flashtube assemblies, each of the flashtube assemblies including:
a xenon flashtube having an output face;
a color separation filter disposed intermediate the output face of the flashtube and the second service cable assembly.

5. The endoscope system of claim 4 wherein the light source comprises first, second and third flashtube assemblies, the color separation filter of the first flashtube assembly is a red filter, the color separation filter of the second flashtube assembly is a green filter and the color separation filter of the third flashtube assembly is a blue filter.

6. The endoscope system of claim 5 wherein each of the flashtube assemblies also includes a light focus optic disposed intermediate the color separation filter and the second service cable assembly.

7. The endoscope system of claim 5 wherein the second service cable assembly comprises:
a conduit segment; and
an input segment including a trifurcated fiber optic conduit having three input ends, a one of the input ends being in optical communication with each of the flashtube assemblies.

8. The endoscope system of claim 7 wherein the trifurcated fiber optic conduit multiplexes the light output of the flashtube assemblies to produce a single light output to the conduit segment.

9. The endoscope system of claim 1 wherein the second service cable assembly comprises:
an optical conduit including
a fiber optic bundle defining a light path and
an infrared filter disposed within the light path; and
an electrical conduit.

10. The endoscope system of claim 9 wherein the light source emits ultra violet light and the optical conduit also includes an ultra violet filter, the ultra violet filter being movable from an imaging position, within the light path, to a intervention position, outside of the light path.

11. The endoscope system of claim 10 wherein the micro-endoscopic device comprises:
an optical probe;
a sensor array; and
a UV filter disposed between the optical probe and the sensor array for protecting the sensor array from the intervention energy.

12. The endoscope system of claim 5 wherein the second service cable assembly comprises:
a conduit segment; and
an input segment including a quad-furcated fiber optic having first, second, third and fourth input ends, the first, second and third input ends being in optical communication with the first, second and third flashtube assemblies, respectively, the fourth input end being in optical communication with the intervention energy source.

13. The endoscope system of claim 12 wherein the intervention energy source is a therapeutic laser unit.

14. The endoscope system of claim 13 wherein the input segment also includes a mechanical fail-safe shutter disposed between the laser unit and the fourth input end, the shutter being in electrical communication with the control module, whereby the control module maintains the shutter in a closed position except when intervention therapy is administered.

15. The endoscope system of claim 13 wherein the micro-endoscopic device comprises:
an optical probe; and
a sensor array having a shutter in electrical communication with the control module, the shutter being disabled in a closed position when intervention therapy is administered.

16. The endoscope system of claim 13 wherein the control module provides sequential control of the flashtube assemblies and the laser unit for imaging and for intervention therapy.

17. The endoscope system of claim 4 wherein the light source comprises first, second, third and fourth flashtube assemblies, the color separation filter of the first flashtube assembly is a red filter, the color separation filter of the second flashtube assembly is a green filter, the color separation filter of the third flashtube assembly is a blue filter, and the color separation filter of the fourth flashtube assembly is in the visible range of the color spectrum.

18. The endoscope system of claim 17 wherein the second service cable assembly comprises:
a conduit segment; and
an input segment including a trifurcated fiber optic conduit and a bifurcated fiber optic conduit, the trifurcated fiber optic conduit having first, second and third input ends in optical communication with the first, second and third flashtube assemblies, respectively, the bifurcated fiber optic conduit having fourth and fifth input ends, the fourth input end being in optical communication with the fourth flashtube assembly and the fifth input end being in optical communication with the intervention energy source.

19. The endoscope system of claim 18 wherein the control module comprises:
a video monitor;
system power supplies; and
a system processing and control electronics unit that activates the fourth flashtube assembly to paint the target area onto the video monitor.

20. The endoscope system of claim 18 wherein the intervention energy source is a therapeutic laser unit and the system further comprises an optical connector disposed between the fifth input end and the laser unit, the optical connector comprising turret-mounted optics providing selection of different laser effects.

21. An endoscope system for providing imaging and intervention therapy comprising:
a control module including
a video monitor and
a control electronics unit
a light source including first, second and third flashtube assemblies, each of the flashtube assemblies including
a xenon flashtube activated by the control electronics unit to emit light and
a band-pass filter passing a narrow frequency band of the light emitted by the flashtube, the band-pass filters of the first, second and third flashtube assemblies each passing a different frequency band of light;
an intervention energy source;
a micro-endoscopic device including
an optical probe and
a sensor array;
a first service cable assembly electrically connecting the control module to the light source and the intervention energy source; and
a second service cable assembly electrically and optically connecting the light source and the intervention energy source to the micro-endoscopic device;
wherein the light source is activated by the control electronics unit to emit a series of light pulses, the second service cable assembly conducts the light pulses to the optical probe, the optical probe directs the light pulses to a target area, light reflected from the target area is collected by the optical probe, the optical probe conducts the reflected light to the sensor array, and the sensor array transmits an image of the target area to the video monitor, and wherein the control electronics unit is operable to activate the intervention energy source to emit intervention energy which is conducted to the target area by the second service cable assembly and the optical probe.

22. An endoscope system for providing imaging and intervention therapy comprising:
a control module;
a light source in electrical communication with the control module:
an intervention energy source in electrical communication with the control module;
a micro-endoscopic device including
an optical probe in optical communication with the light source and the intervention energy source, and
a sensor array in optical communication with the optical probe and in electrical communication with the control module;
wherein the light source is activated by the control module to emit a series of light pulses, the optical probe directs the light pulses to a target area, light reflected from the target area is collected by the optical probe, the optical probe conducts the reflected light to the sensor array, and the sensor array transmits an image of the target area to the control module, and wherein the control module is operable to activate the intervention energy source to emit intervention energy which is conducted to the target area by the optical probe.
